(19) United States
(12) Patent Application Publication
(10) Pub. No.: US 2024/0382703 A1
(43) Pub. Date: Nov. 21, 2024

(54) INHALER AUTHORISATION APPARATUS AND AN INHALER INCLUDING SUCH AN APPARATUS

(71) Applicant: KINDEVA DRUG DELIVERY L.P., Woodbury, MN (US)

(72) Inventor: John P. BUNTING, Castle Donington (GB)

(21) Appl. No.: 18/694,108

(22) PCT Filed: Sep. 21, 2022
(86) PCT No.: PCT/US2022/044218

§ 371 Date: Mar. 21, 2024

(30) Foreign Application Priority Data
Sep. 22, 2021 (GB) 2113478.8

(51) Int. Cl. A61M 15/00

(52) U.S. Cl. CPC A61M 15/0001; A61M 2205/276; A61M 2205/3327

(57) ABSTRACT

An inhaler (100) has an actuator (118) configured to interact with a container (110) to selectively release a substance for inhalation. A controller (112) is provided and configured to switch the inhaler between a locked and an unlocked condition. A motion sensor (114) is attached to the inhaler. The controller (112) is configured to store a predetermined motion sequence, and upon detection of the predetermined motion sequence by the motion sensor, the controller is configured to switch the inhaler from the locked condition to the unlocked condition.

### TECHNICAL FIELD

The present disclosure relates to the field of inhalers. More specifically, the present disclosure relates to the field of apparatus for authorising use of an inhaler to dispense medicament to an authorised user.

### BACKGROUND ART

In general, an inhaler is a device that is configured to produce a dose of an aerosolised substance for entrainment into the mouth or nose of a user, typically accompanied by the act of inhalation. Typically, but not exclusively, inhalers are medical inhalers, i.e., those configured to dispense aerosolised medicament for the treatment of a variety of conditions such as asthma or COPD. Other types of therapeutic inhalers are available, for example, those containing alternative medicines and natural ingredients, but the overarching principle is that therapeutic inhalers offer a benefit to the human or animal body.

Inhalers are provided in many forms, such as dry powder inhalers (DPI) and pressurised metered dose inhalers (pMDIs). In the past, such inhalers have been provided with a container for the medicament and an actuator/dosing mechanism by which a dose of medicament can be released for inhalation by the user.

More recently, technology has been introduced to inhalers both to increase their effectiveness and usability. For example, electrically powered actuation has become more common, as has electronic functionality such as dose counters.

It is desirable to prevent unauthorised use of an inhaler. For example, unauthorised access to medicaments by children is highly undesirable. Inhalers may also contain controlled substances which are prone to misuse such as opioids.

WO2019/157208 describes an electronic metered-dose inhaler (MDI) system. Security features validate the user using an associated mobile app to verify the user's identity. The app and inhaler communicate wirelessly. The inhaler includes an accelerometer which verifies that the inhaler has been properly shaken (and the contents appropriately agitated) before medicament dispensing is authorised. This system is reliant upon the presence, battery status, and communication with, a mobile device having the appropriate app installed.

WO2011/114355 describes an MDI with a locking means to lock the actuator. The actuator can be unlocked upon shaking the device, allowing the MDI to deliver the medicament once rendered homogeneous. This device is intended to ensure that the contents are properly homogenised before dosing.

WO2020/102229 describes a device in which actuation of a canister may be blocked until the solution is properly shaken. An accelerometer determines how hard the device has been shaken.

### SUMMARY OF DISCLOSURE

This disclosure includes without limitation the following clauses:

Clause 1: An inhaler for delivery of a therapeutic substance, the inhaler including an actuator configured to interact with a container to selectively release the therapeutic substance for inhalation; and a controller configured to switch the inhaler between a locked condition in which release of the substance from the container is either inhibited, or permitted within at least one locked constraint; and an unlocked condition, in which release of the substance from the container is either permitted, or permitted within at least one unlocked constraint being less restrictive than the at least one locked constraint. The inhaler further includes a motion sensor attached to the inhaler. The controller is configured to store a predetermined motion sequence. Further, upon detection of the predetermined motion sequence by the motion sensor, the controller is configured to switch the inhaler from the locked condition to the unlocked condition.

Advantageously, one or more embodiments of the present disclosure can provide a way of enabling a user input for security purposes that does not require an external device or complex equipment such as a keyboard or fingerprint scanner.

Clause 2: An inhaler according to Clause 1, where the motion sensor is configured to sense motion in at least two directions. By “directions” we mean along different axes at an angle to each other rather than forward and reverse along the same axis.

Clause 3: An inhaler according to Clause 2, where the motion sensor is configured to sense motion in three directions.

Clause 4: An inhaler according to any preceding Clause, having a user-accessible configuration mode in which the user moves the inhaler in the predetermined motion sequence which is detected by the motion sensor and recorded and stored by the controller.

Clause 5: An inhaler according to Clause 4, in which the user must move the inhaler in the predetermined motion sequence at least twice to record the predetermined motion sequence.

Clause 6: An inhaler according to Clause 4 or 5, where the controller is configured to assess the input predetermined motion sequence against at least one predetermined criterion, and if the input predetermined motion sequence does not meet the at least one predetermined criterion, it is rejected.

Clause 7: An inhaler according to Clause 6, where the at least one predetermined criterion includes at least two-dimensional motion.

Clause 8: An inhaler according to Clause 7, where the at least one predetermined criterion includes three-dimensional motion.

Clause 9: An inhaler according to any preceding Clause, where the predetermined motion sequence includes one or more of the following: a minimum number of reversals of direction in each direction: is of a minimum duration: does not exceed a maximum duration; or varies in a specific manner over time. By “varies in a specific manner over time” we mean that the speed of movement of the inhaler, and/or the time intervals between movements is included in the motion sensing.

Clause 10: An inhaler according to any preceding Clause, where in the locked condition release of the substance from the container is inhibited; and in the unlocked condition, release of the substance from the container is permitted.

Clause 11: An inhaler according to any of Clauses 1 to 9, where in the locked condition, release of the substance from the container is inhibited: in the unlocked condition, release of the substance from the container is permitted within at least one unlocked constraint.

Clause 12: An inhaler according to any of Clauses 1 to 9, where in the locked condition, release of the substance from the container is inhibited within at least one locked constraint; and in the unlocked condition, release of the substance from the container is permitted within at least one unlocked constraint being less restrictive than the at least one locked constraint.

Clause 13: An inhaler according to any preceding Clause, where the actuator is electrically powered to selectively release the substance for inhalation, and where the controller electronically controls the actuator when switching between the locked and unlocked conditions.

Clause 14: An inhaler according to any of Clauses 1 to 12, including a lock configured to inhibit actuation of the actuator to selectively release the substance for inhalation, where the controller electronically controls the lock when switching between the locked and unlocked conditions.

Clause 15: A method of using an inhaler for delivery of a therapeutic substance, the method including providing an inhaler that includes an actuator configured to interact with a container to selectively release the therapeutic substance for inhalation; and a controller configured to switch the inhaler between a locked condition in which release of the substance from the container is either inhibited, or permitted within at least one locked constraint; and an unlocked condition in which release of the substance from the container is either permitted, or permitted within at least one unlocked constraint being less restrictive than the at least one locked constraint. The inhaler further includes a motion sensor attached to the inhaler. The controller is configured to store a predetermined motion sequence. The method further includes moving the inhaler in a motion sequence: determining by the controller whether the motion sequence matches the predetermined motion sequence; and, if the motion sequence matches the predetermined motion sequence, switching the inhaler from the locked condition to the unlocked condition.

Clause 16: A method of using an inhaler for delivery of a therapeutic substance, including providing an inhaler that includes an actuator configured to interact with a container to selectively release the therapeutic substance for inhalation; and a controller configured to switch the inhaler between a locked condition in which release of the substance from the container is either inhibited, or permitted within at least one locked constraint; and an unlocked condition, in which release of the substance from the container is either permitted, or permitted within at least one unlocked constraint being less restrictive than the at least one locked constraint. The inhaler further includes a motion sensor attached to the inhaler. The controller is configured to store a predetermined motion sequence. The method further includes moving the inhaler in a motion sequence: recording the motion sequence using the controller: storing the motion sequence as a predetermined motion sequence; and switching the inhaler from the locked condition to the unlocked condition upon movement of the inhaler in the predetermined motion sequence.

Clause 17: A method of using an inhaler according to Clause 16, including assessing the input motion sequence against at least one predetermined criterion; and if the input motion sequence does not meet the at least one predetermined criterion, rejecting the input motion sequence.

Clause 18: A method of using an inhaler according to Clause 17, including moving the inhaler in the motion sequence for a second time: recording the second motion sequence using the controller; assessing the second input motion sequence against the first input motion sequence; and if the first and second input motion sequences do not match, rejecting the first and second input motion sequences.

Clause 19: A method according to any of Clauses 17 to 18, where the at least one predetermined criterion includes at least two-dimensional motion.

Clause 20: A method according to Clause 19, where the at least one predetermined criterion includes three-dimensional motion.

Clause 21: A method according to any of Clauses 16 to 20, where the predetermined motion sequence includes one or more of the following: a minimum number of reversals of direction in each direction: is of a minimum duration: does not exceed a maximum duration; or varies in a specific manner over time.

### BRIEF DESCRIPTION OF DRAWINGS

Embodiments of the present disclosure will now be described with reference to the following figures in which:

FIG. 1 is a schematic view of a first inhaler according to the present disclosure:

FIG. 2a is a flow chart of a setup sequence according to the present disclosure:

FIG. 2b is a flow chart of a use sequence according to the present disclosure;

FIG. 3 illustrates side and front views of an inhaler showing the axes referred to in the present disclosure; and

FIGS. 4a to 4d are various schematic views of motion sequences, where FIG. 4a is a schematic view of motion in the positive and negative Z directions: FIG. 4b is a schematic view of motion in the positive and negative X, Y, and Z directions; FIG. 4c is a schematic view of a figure eight motion; and FIG. 4d is a schematic view of a motion in a pattern of someone's initials.

### DESCRIPTION OF THE FIRST EMBODIMENT

### Configuration

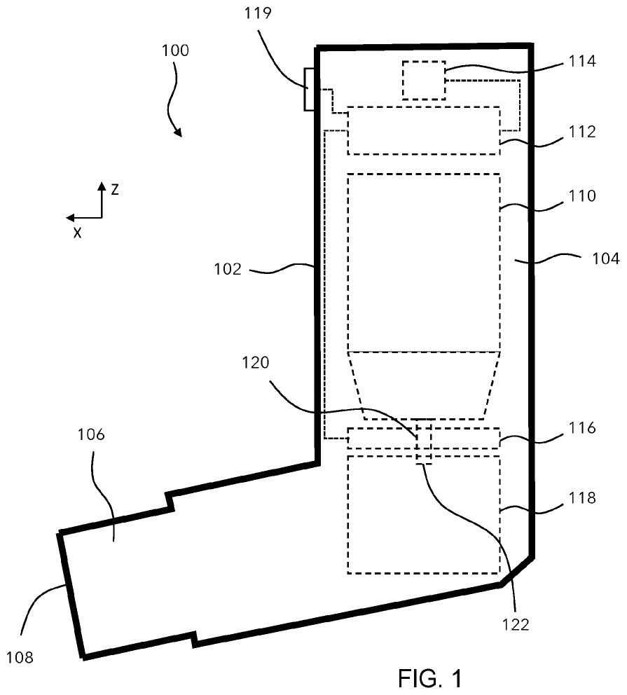

Referring to FIG. 1, a pressurised metered-dose inhaler (pMDI) 100 includes a body 102 having a canister-receiving portion 104 and a mouthpiece 106 with an outlet orifice 108. The body 102 in this embodiment is constructed from a moulded plastics material, although other materials are possible.

The pMDI 100 body 102 contains a pressurised canister (i.e., container) 110, a controller 112, a movement sensor 114, a locking mechanism (e.g., lock) 116, an actuator 118, and a user display 119.

The canister 110 contains a pressurised medicament (i.e., therapeutic substance) and propellant and includes a metering valve 120 which provides selective fluid communication between a valve outlet 122 and the inside of the canister 110. The metering valve 120 has a closed state in which no communication is permitted, and an open state in which a predetermined volume of medicament is released to the valve outlet 122.

The controller 112 can include a memory, processor, an input/output module as well as a power source (e.g., a battery).

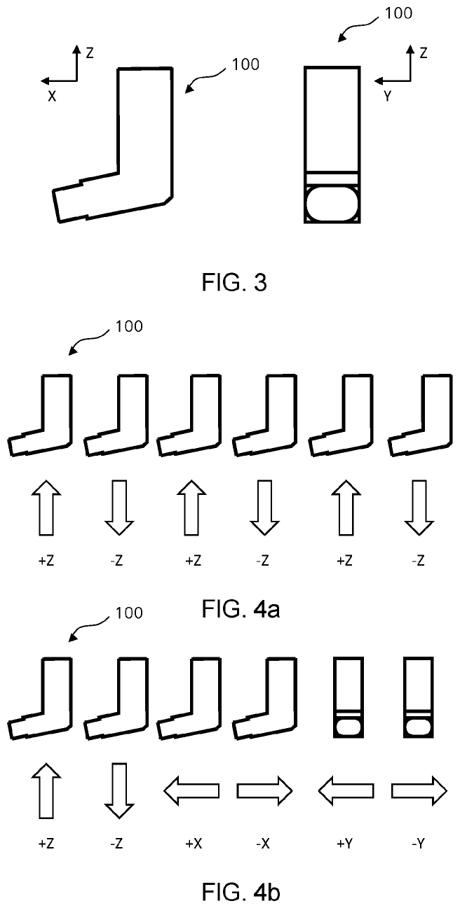

The movement sensor 114 can be a tri-axial accelerometer which is configured to detect movement in three cartesian axes of the pMDI 100 (X, Y, Z shown in FIG. 3).

The actuator 118 can be configured to interact with the container 110 to selectively release the therapeutic substance for inhalation. In one or more embodiments, the actuator 118 is configured to receive the valve 120 and selectively release a dose of the therapeutic substance from within the container 110. The locking mechanism 116 is positioned between the container 110 and actuator 118 and is configured to either permit or inhibit actuation of the valve 120 to release a dose of therapeutic substance to the orifice 108. Any suitable technique can be utilized to selectively release the dose. In one or more embodiments, the actuator 118 can be electrically powered to selectively release the therapeutic substance for inhalation. In one or more embodiments, the controller 112 can be configured to electronically control the actuator 118 when switching between the locked and unlocked conditions.

The user display 119 in this embodiment is a small screen capable of displaying short messages to the user.

The controller 112 is in communication with the movement sensor 114, the locking mechanism 116 (e.g., lock) and the user display 119. The controller 112 is configured to receive a motion input from the movement sensor 114, to analyse the motion input, and to switch the locking mechanism 116 from a locked condition to an unlocked condition depending on the output of the analysis. The controller 112 is also configured to control the messages provided via the display 119. In the locked condition, release of the substance from the container 110 is either inhibited, or permitted within at least one locked constraint. For example, in one or more embodiments, a locked constraint can include a dosing protocol, where the locking mechanism 116 will not be switched from the locked condition to the unlocked condition unless a sufficient period of time has elapsed since the last dose was administered. Further, in the unlocked condition, release of the substance from the container 110 is either permitted, or permitted within at least one unlocked constraint being less restrictive than the at least one locked constraint. For example, in one or more embodiments, an unlocked constraint can include a time out feature, where when the inhaler is unlocked it must be used in a certain time period.

### Use

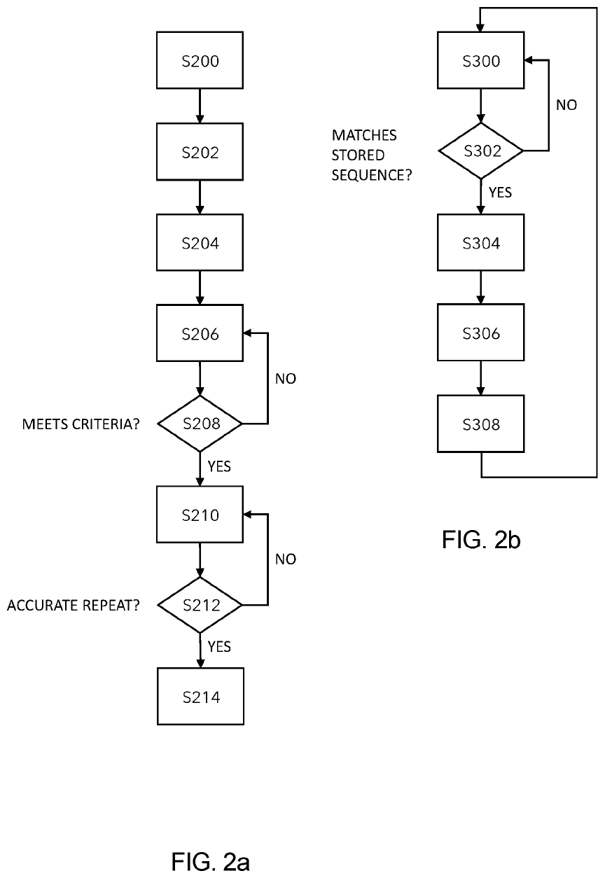

Referring to FIG. 2a, one embodiment of a setup/configuration method for the pMDI 100 is shown. At step S200 an authorised user receives the pMDI 100. At this stage, the lock/unlock protocol controlled by the controller 112 is disabled.

At step S202 the user activates the inhaler by pressing a power on control (not shown) such as a button.

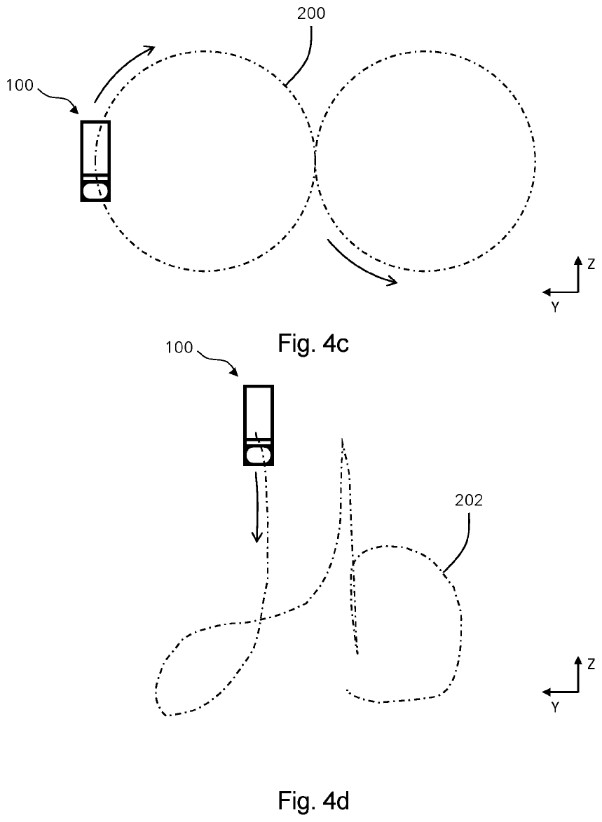

At step S204, a user-accessible configuration mode is activated, where the user is prompted to record a movement sequence by the display 119. In response to the prompt, at step S206 the user moves the inhaler in at least one direction in a desired sequence. Turning to FIG. 3, the cartesian coordinates of the pMDI 100 are shown. FIGS. 4a and 4b show two examples of simple movement patterns involving back and forth motion in at least one direction. FIG. 4a shows a simple three phase movement in the positive and negative Z directions only, and FIG. 4b shows a simple three phase movement in the positive and negative X, Y, Z directions.

FIGS. 4c and 4d show more complex movements involving movement in at least two directions simultaneously (i.e., in two- or three-dimensional space). FIG. 4c shows a motion sequence in the form of a “figure eight” motion path 200 in the ZY plane. FIG. 4d shows a motion sequence in the form of a motion path 202 in the ZY plane describing the user's initials “jb.” It will be understood that the user may choose to input one or more of the following, non-exhaustive list of motion paths and combinations thereof:
Symbols;
Letters; or
Pictures.

As well as recording the motion sequence in three-dimensional space, the controller 112 may also record the timing of the sequence. For example, the user may add one or more of the following time-dependent characteristics to the motion sequence:
Adjustments in frequency;
Pauses;
Changes in speed; or
Predetermined rhythms, for example, in time to a memorised tune.

At step S208, the controller 112 is configured to determine whether the motion sequence meets at least one predetermined criteria to provide a predetermined motion sequence. The criteria may include at least one of the following:
The motion sequence is sufficiently complex, for example:
   Includes motion in at least two directions (X, Y, Z);
   Includes a minimum number of reversals of direction in each axis;
   Is of a minimum duration; or
   Does not exceed a maximum duration.
The motion sequence is sufficient to agitate and/or homogenise the contents. For example, the motion sequence can include a predetermined number of reversals in the Z direction.

Moving to step S210, if the motion sequence meets the predetermined criteria at step S208, the controller 112 provides the user with an indication that the user should repeat the sequence via the display 119. If the motion sequence does not meet the criteria, the controller 112 requests that the user input a new sequence, i.e., the input motion sequence is rejected.

In one or more embodiments, the controller 112 can require that the user move the inhaler in the predetermined motion sequence at least twice to record the predetermined motion sequence. For example, at step S210 the user repeats the motion sequence a second or subsequent time. The controller 112 can record the second motion sequence. At step S212, the controller 112 is configured to determine whether the motion sequence has been repeated successfully. In one or more embodiments, the controller 112 can be configured to determine if the motion sequence has been repeated successfully if the first input motion matches the second input motion. If the first and second input motions do not match, then the controller 112 can be configured to reject the first and second input motion sequences. If the first and second input motions match, at step S214 the predetermined motion sequence is stored and the user is told via the display 119 that input was successful.

Referring to FIG. 2b, one embodiment of a use method for the pMDI 100 is shown. At step S300 a user moves the inhaler 100 in a motion sequence. At step S302 the controller 112 checks the motion sequence against the stored predetermined motion sequence from FIG. 2a. If the input motion sequence matches the stored predetermined motion sequence, at step S304 the inhaler 100 is switched from the locked condition to the unlocked condition by the controller 112 and the inhaler can be used. If not, the user is presented with an error message and returned to step S300 to try again.

At step S306 the device is actuated, which actuation is detected by the controller 112, and the device is locked once more at step S308.

### Variations

The following variations to the embodiments described herein fall within the scope of the present disclosure.

The user display 119 may be a simple light, a complex display, or may be provided on a separate device (e.g., a mobile device such as a mobile phone) in communication with the inhaler 100 via e.g., Bluetooth®.

A “master” sequence may be stored within the controller 112 that allows the inhaler 100 to be reset should the user forget their motion sequence.

The inhaler 100 may be configured to permit a predetermined number of doses, or doses per time period (e.g., hour or day) without having to be unlocked. For example, one dose per hour may be permitted, after which the inhaler 100 is locked, and can only be unlocked with the above motion sequence.

The inhaler 100 may be provided with a “time out” feature in which at step S308 it is locked after a predetermined time period (e.g., 1 minute) if it is not used. The time out may be overridden by a single use, a predetermined number of uses, or may allow unlimited multiple doses during the predetermined time period.

The inhaler 100 may inhibit use once an integrated dose counter has reached a predetermined limit, e.g., if the maximum dose is about to be exceeded, or if the dose counter indicates that the canister is exhausted.

The inhaler 100 may communicate the motion sequence to a mobile device, where it can be visualised (similar to FIGS. 4c and 4d) as a reminder to the user.

The disclosure may be implemented on inhaler types other than pressurised metered-dose inhalers (pMDIs).

Instead of a locking mechanism 116, the inhaler 100 may be provided with an electronically controlled actuation mechanism that can only be activated when unlocked by the correct motion sequence (i.e., it is electronically locked).

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. An inhaler for delivery of a therapeutic substance, the inhaler comprising:
an actuator configured to interact with a container to selectively release the therapeutic substance for inhalation;
a controller configured to switch the inhaler between:
a locked condition in which release of the substance from the container is either inhibited, or permitted within at least one locked constraint; and,
an unlocked condition, in which release of the substance from the container is either permitted, or permitted within at least one unlocked constraint being less restrictive than the at least one locked constraint; and
a motion sensor attached to the inhaler;
wherein:
the controller is configured to store a predetermined motion sequence; and
upon detection of the predetermined motion sequence by the motion sensor, the controller is configured to switch the inhaler from the locked condition to the unlocked condition.

2. An inhaler according to claim 1, wherein the motion sensor is configured to sense motion in at least two directions.

3. (canceled)

4. An inhaler according to claim 1, having a user-accessible configuration mode in which the user moves the inhaler in the predetermined motion sequence which is detected by the motion sensor and recorded and stored by the controller.

5. An inhaler according to claim 4, in which the user must move the inhaler in the predetermined motion sequence at least twice to record the predetermined motion sequence.

6. An inhaler according to claim 4, wherein the controller is configured to assess the input predetermined motion sequence against at least one predetermined criterion, and if the input predetermined motion sequence does not meet the at least one predetermined criterion, it is rejected.

7. An inhaler according to claim 6, wherein the at least one predetermined criterion includes at least two-dimensional motion.

8. An inhaler according to claim 7, wherein the at least one predetermined criterion includes three-dimensional motion.

9. An inhaler according to claim 1, wherein the predetermined motion sequence includes one or more of the following:
a minimum number of reversals of direction in each direction;
is of a minimum duration;
does not exceed a maximum duration; or
varies in a specific manner over time.

10. An inhaler according to claim 1, wherein:
in the locked condition release of the substance from the container is inhibited; and
in the unlocked condition, release of the substance from the container is permitted.

11. An inhaler according to claim 1, wherein:
in the locked condition, release of the substance from the container is inhibited; and
in the unlocked condition, release of the substance from the container is permitted within at least one unlocked constraint.

12. An inhaler according to claim 1, wherein:
in the locked condition, release of the substance from the container is inhibited within at least one locked constraint; and
in the unlocked condition, release of the substance from the container is permitted within at least one unlocked constraint being less restrictive than the at least one locked constraint.

13. An inhaler according to claim 1, wherein the actuator is electrically powered to selectively release the substance for inhalation, and wherein the controller electronically controls the actuator when switching between the locked and unlocked conditions.

14. An inhaler according to claim 1, comprising a lock configured to inhibit actuation of the actuator to selectively release the substance for inhalation, wherein the controller electronically controls the lock when switching between the locked and unlocked conditions.

15. A method of using an inhaler for delivery of a therapeutic substance, the method comprising:
providing an inhaler comprising:
an actuator configured to interact with a container to selectively release the therapeutic substance for inhalation;
a controller configured to switch the inhaler between:
a locked condition in which release of the substance from the container is either inhibited, or permitted within at least one locked constraint; and
an unlocked condition in which release of the substance from the container is either permitted, or permitted within at least one unlocked constraint being less restrictive than the at least one locked constraint; and
a motion sensor attached to the inhaler;
wherein the controller is configured to store a predetermined motion sequence;
moving the inhaler in a motion sequence;
determining by the controller whether the motion sequence matches the predetermined motion sequence; and
if the motion sequence matches the predetermined motion sequence, switching the inhaler from the locked condition to the unlocked condition.

16. A method of using an inhaler for delivery of a therapeutic substance, the method comprising:
providing an inhaler comprising:
an actuator configured to interact with a container to selectively release the therapeutic substance for inhalation;
a controller configured to switch the inhaler between:
a locked condition in which release of the substance from the container is either inhibited, or permitted within at least one locked constraint; and
an unlocked condition, in which release of the substance from the container is either permitted, or permitted within at least one unlocked constraint being less restrictive than the at least one locked constraint; and
a motion sensor attached to the inhaler;
wherein the controller is configured to store a predetermined motion sequence;
moving the inhaler in a motion sequence;
recording the motion sequence using the controller;
storing the motion sequence as a predetermined motion sequence; and
switching the inhaler from the locked condition to the unlocked condition upon movement of the inhaler in the predetermined motion sequence.

17. A method of using an inhaler according to claim 16, comprising:
assessing the input motion sequence against at least one predetermined criterion; and
if the input motion sequence does not meet the at least one predetermined criterion, rejecting the input motion sequence.

18. A method of using an inhaler according to claim 17, comprising:
moving the inhaler in the motion sequence for a second time;
recording the second motion sequence using the controller;
assessing the second input motion sequence against the first input motion sequence;
if the first and second input motion sequences do not match, rejecting the first and second input motion sequences.

19. A method according to claim 17, wherein the at least one predetermined criterion includes at least two-dimensional motion.

20. A method according to claim 19, wherein the at least one predetermined criterion includes three-dimensional motion.

21. A method according to claim 16, wherein the predetermined motion sequence comprises one or more of the following:
a minimum number of reversals of direction in each direction;
is of a minimum duration;
does not exceed a maximum duration; or
varies in a specific manner over time.
